(19) United States
(12) Patent Application Publication
(10) Pub. No.: US 2024/0325278 A1
(43) Pub. Date: Oct. 3, 2024

(54) Personal Care Compositions

(71) Applicant: Colgate- Palmolive Company, New York, NY (US)

(72) Inventors: Nan LI, Piscataway, NJ (US); Hongwei Shen, Holmdel, NJ (US); Shijian Wu, Piscataway, NJ (US); Andrei Potanin, Hillsborough, NJ (US)

(73) Assignee: Colgate- Palmolive Company, New York, NY (US)

(21) Appl. No.: 18/619,876

(22) Filed: Mar. 28, 2024

Related U.S. Application Data
(60) Provisional application No. 63/493,415, filed on Mar. 31, 2023.

(51) Int. Cl. A61K 8/73; A61K 8/44; A61K 8/49; A61K 8/60; A61Q 5/02; A61Q 5/12; A61Q 19/10

(52) U.S. Cl. CPC A61K 8/73; A61K 8/442; A61K 8/4946; A61K 8/604; A61K 8/731; A61Q 5/02; A61Q 5/12; A61Q 19/10

(57) ABSTRACT

Described herein is a viscosity control composition comprising a surfactant blend comprising a biosurfactant, an anionic surfactant, a zwitterionic surfactant comprising sodium lauroamphoacetate, a gum comprising carrageenan, and a liquid carrier.

### CROSS-REFERENCE TO RELATED APPLICATIONS

This application claims the benefit of priority from U.S. Patent Application No. 63/493,415, filed Mar. 31, 2023, titled Personal Care Compositions, the content of which is hereby incorporated herein in its entirety, for all purposes.

### BACKGROUND

Surfactants are utilized in personal care and home care products to impart foaming properties. The use of surfactants also raises concerns about environmental influences and dermatological issues. While biosurfactants may be used for sustainability and more mild interactions, such biosurfactants also have more complicated structures than conventional surfactants and present a challenge to control the impact of performance of biosurfactant micelles. Therefore, a need exists to improve on such biosurfactants being used in such cleansing liquids.

### BRIEF SUMMARY

The present invention is directed to a viscosity control composition comprising a surfactant blend comprising a biosurfactant; an anionic surfactant; a zwitterionic surfactant comprising sodium lauroamphoacetate; a gum comprising carrageenan; and a liquid carrier.

Other embodiments of the present invention include a viscosity control composition comprising: a surfactant blend comprising a biosurfactant; an anionic surfactant; a zwitterionic surfactant; a gum comprising carrageenan; and a liquid carrier; wherein the anionic surfactant and the biosurfactant are present in a weight ratio ranging from about 1:20 to about 20:1; wherein the zwitterionic surfactant and the biosurfactant are present in a weight ratio ranging from about 1:10 to about 10:1; and wherein the gum and the biosurfactant are present in a weight ratio ranging from about 1:30 to about 30:1.

Other embodiments of the present invention include a viscosity control composition comprising: a rhamnolipid; sodium lauroyl sarcosinate; sodium lauroamphoacetate; carrageenan; and a liquid carrier.

Other embodiments of the present invention include a personal care composition comprising: the aforementioned viscosity control composition; and a personal care ingredient; wherein the personal care composition is a liquid hand soap, shower gel, shampoo, conditioner, body wash, facial cleanser or skin cleanser.

Other embodiments of the present invention include a method of manufacturing a personal care product having viscosity control, the method comprising: blending together a viscosity control composition as described herein with a personal care additive to form a personal care composition; adding a predetermine volume of the personal care composition to a container to form the personal care product.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

A list of non-limiting, example embodiments is provided below:

In accordance with an embodiment 1, provided is a viscosity control composition comprising: a surfactant blend comprising: a biosurfactant; an anionic surfactant; a zwitterionic surfactant comprising sodium lauroamphoacetate; a gum comprising carrageenan; and a liquid carrier.

In accordance with an embodiment 2, provided is a viscosity control composition according to embodiment 1, wherein the biosurfactant comprises rhamnolipid.

In accordance with an embodiment 3, provided is a viscosity control composition according to any one of embodiments 1 to 2, wherein the biosurfactant is present in an amount ranging from about 0.01 wt. % to about 13 wt. % based on the weight of the viscosity control composition.

In accordance with an embodiment 4, provided is a viscosity control composition according to any one of embodiments 1 to 3, wherein the anionic surfactant comprises an amine-containing compound.

In accordance with an embodiment 5, provided is a viscosity control composition according to any one of embodiments 1 to 4, wherein the anionic surfactant comprises sodium lauroyl sarcosinate.

In accordance with an embodiment 6, provided is a viscosity control composition according to any one of embodiments 1 to 5, wherein the anionic surfactant is present in an amount ranging from about 0.1 wt. % to about 13.0 wt. % based on the weight of the viscosity control composition.

In accordance with an embodiment 7, provided is a viscosity control composition according to any one of embodiments 1 to 6, wherein the zwitterionic surfactant is present in an amount ranging from about 1.0 wt. % to about 24.0 wt. % based on the weight of the viscosity control composition.

In accordance with an embodiment 8, provided is a viscosity control composition according to any one of embodiments 1 to 7, wherein the gum is present in an amount ranging from about 0.1 wt. % to about 5.0 wt. % based on the weight of the viscosity control composition.

In accordance with an embodiment 9, provided is a viscosity control composition according to any one of embodiments 1 to 8, wherein the gum consists of carrageenan.

In accordance with an embodiment 10, provided is a viscosity control composition according to any one of embodiments 1 to 9, wherein the anionic surfactant and the biosurfactant are present in a weight ratio ranging from about 0.6:1 to about 2:1.

In accordance with an embodiment 11, provided is a viscosity control composition according to any one of embodiments 1 to 10, wherein the zwitterionic surfactant and the biosurfactant are present in a weight ratio ranging from about 5:1 to about 10:1.

In accordance with an embodiment 12, provided is a viscosity control composition according to any one of embodiments 1 to 11, wherein the gum and the biosurfactant are present in a weight ratio ranging from about 0.5:1 to about 2:1.

In accordance with an embodiment 13, provided is a viscosity control composition according to any one of embodiments 1 to 12, wherein the liquid carrier comprises water.

In accordance with an embodiment 14, provided is a viscosity control composition according to any one of embodiments 1 to 13, wherein the surfactant blend, the gum, and the liquid carrier total to about 100 wt. % of the viscosity control composition.

In accordance with an embodiment 15, provided is a viscosity control composition comprising: a surfactant blend comprising: a biosurfactant; an anionic surfactant; a zwitterionic surfactant; a gum comprising carrageenan; and a liquid carrier; wherein the anionic surfactant and the biosurfactant are present in a weight ratio ranging from about 0.6:1 to about 2:1; wherein the zwitterionic surfactant and the biosurfactant are present in a weight ratio ranging from about 5:1 to about 10:1; and wherein the gum and the biosurfactant are present in a weight ratio ranging from about 0.5:1 to about 2:1.

In accordance with an embodiment 16, provided is a viscosity control composition according to embodiment 15, wherein the biosurfactant comprises rhamnolipid.

In accordance with an embodiment 17, provided is a viscosity control composition according to any one of embodiments 15 to 16, wherein the biosurfactant is present in an amount ranging from about 0.1 wt. % to about 3 wt. % based on the weight of the viscosity control composition.

In accordance with an embodiment 18, provided is a viscosity control composition according to any one of embodiments 15 to 17, wherein the anionic surfactant comprises an amine-containing compound.

In accordance with an embodiment 19, provided is a viscosity control composition according to any one of embodiments 15 to 18, wherein the anionic surfactant comprises sodium lauroyl sarcosinate.

In accordance with an embodiment 20, provided is a viscosity control composition according to any one of embodiments 15 to 19, wherein the anionic surfactant is present in an amount ranging from about 0.5 wt. % to about 3.5 wt. % based on the weight of the viscosity control composition.

In accordance with an embodiment 21, provided is a viscosity control composition according any one of embodiments 15 to 20, wherein the zwitterionic surfactant comprising sodium lauroamphoacetate.

In accordance with an embodiment 22, provided is a viscosity control composition according to any one of embodiments 15 to 21, wherein the zwitterionic surfactant is present in an amount ranging from about 2.0 wt. % to about 12.0 wt. % based on the weight of the viscosity control composition.

In accordance with an embodiment 23, provided is a viscosity control composition according to any one of embodiments 15 to 22, wherein the gum is present in an amount ranging from about 0.1 wt. % to about 5.0 wt. % based on the weight of the viscosity control composition.

In accordance with an embodiment 24, provided is a viscosity control composition according to any one of embodiments 15 to 23, wherein the gum consists of carrageenan.

In accordance with an embodiment 25, provided is a viscosity control composition according to any one of embodiments 15 to 24, wherein the liquid carrier comprises water.

In accordance with an embodiment 26, provided is a viscosity control composition according to any one of embodiments 15 to 25, wherein the surfactant blend, the gum, and the liquid carrier total to about 100 wt. % of the viscosity control composition.

In accordance with an embodiment 27, provided is a viscosity control composition comprising: a rhamnolipid; sodium lauroyl sarcosinate; sodium lauroamphoacetate; carrageenan; and a liquid carrier.

In accordance with an embodiment 28, provided is a viscosity control composition according to embodiment 27, wherein the rhamnolipid is present in an amount ranging from about 0.1 wt. % to about 3 wt. % based on the weight of the viscosity control composition.

In accordance with an embodiment 29, provided is a viscosity control composition according to any one of embodiments 27 to 28, wherein the sodium lauroyl sarcosinate is present in an amount ranging from about 0.5 wt. % to about 3.5 wt. % based on the weight of the viscosity control composition.

In accordance with an embodiment 30, provided is a viscosity control composition according to any one of embodiments 27 to 29, wherein the sodium lauroamphoacetate is present in an amount ranging from about 2.0 wt. % to about 12.0 wt. % based on the weight of the viscosity control composition.

In accordance with an embodiment 31, provided is a viscosity control composition according to any one of embodiments 27 to 30, wherein the carrageenan is present in an amount ranging from about 0.1 wt. % to about 5.0 wt. % based on the weight of the viscosity control composition.

In accordance with an embodiment 32, provided is a viscosity control composition according to any one of embodiments 27 to 31, wherein the liquid carrier comprises water.

In accordance with an embodiment 33, provided is a viscosity control composition according to any one of embodiments 27 to 32, wherein the rhamnolipid, the sodium lauroyl sarcosinate, the sodium lauroamphoacetate, the carrageenan, and the liquid carrier total to about 100 wt. % of the viscosity control composition.

In accordance with an embodiment 34, provided is a personal care composition comprising: the viscosity control composition according to any one of embodiments 1 to 33; and a personal care ingredient; wherein the personal care composition is a bath soap, shower gel, shampoo, conditioner, body wash, or skin cleanser.

In accordance with an embodiment 35, provided is a viscosity control composition according to embodiment 34, wherein the personal care ingredient comprises one or more of a dye, fragrance, and combinations thereof.

In accordance with an embodiment 36, provided is a method of manufacturing a personal care product having viscosity control, the method comprising: blending together a viscosity control composition according to any one of embodiments 1 to 33 with personal care ingredient to form a personal care composition; and adding a predetermine volume of the personal care composition to a container to form the personal care product.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention.

Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

Unless otherwise specified, all percentages and amounts expressed herein and elsewhere in the specification should be understood to refer to percentages by weight. The amounts given are based on the active weight of the material. According to the present application, the term “about” means+/−10% of the reference value. According to the present application, the term “substantially free” less than about 0.1 wt. % based on the total of the referenced value.

As used herein, the use of a compound comprising several isomers or stereoisomers includes all the isomeric forms of that compound. When referring to chemical structures, and names, the symbols “C”, “H”, and “O” mean carbon, hydrogen, and oxygen, respectively. The symbols “—”, “═” and “δ” mean single bond, double bond, and triple bond, respectively. For readability purposes, the chemical functional groups are in their adjective form; for each of the adjective, the word “group” is assumed. For example, the adjective “alkyl” without a nouns thereafter, should be read as “an alkyl group.”

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

All components and elements positively set forth in this disclosure can be negatively excluded from the claims. In other words, the personal care compositions of the instant disclosure can be free or essentially free of all components and elements positively recited throughout the instant disclosure. In some instances, the personal care compositions of the present disclosure may be substantially free of non-incidental amounts of the ingredient(s) or compound(s) described herein. A non-incidental amount of an ingredient or compound is the amount of that ingredient or compound that is added into the personal care composition by itself. For example, a personal care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the personal care composition includes both components (or the composition includes more than two components that overlap), an overlapping compound does not represent more than one component. For example, caprylyl glycol may be characterized as both a humectant and a preservative. If a particular personal care composition includes both a humectant and a preservative, caprylyl glycol will serve only as either a humectant or a preservative—not both.

As used herein, the terms “composition” and “formulation” can be used interchangeably within the specification.

Aspects of the invention are directed to viscosity control composition. Aspects of the invention are further directed to personal care composition comprising the viscosity control composition.

The viscosity control composition may comprise a surfactant blend. The surfactant blend may comprise 2 or more surfactants, e.g., 3, 4, 5, 6, 7, 8, 9, 10 surfactants, or a range formed therefrom. For example, the oral care composition may comprise 2 to 6, 2 to 5, 2 to 4, 2 or 3; from 3 to 6, or 3 or 4 surfactants. The surfactant blend may comprise a first surfactant, a second surfactant, and a third surfactant. In some embodiments, the viscosity control composition comprises a surfactant blend of two or more surfactants, wherein at least one surfactant is biosurfactant. For instance, the surfactant blend may comprise two or more surfactants, with at least one of the surfactants being a biosurfactant and at least one of the surfactants being an anionic surfactant (e.g., a non-sulfate based anionic surfactant), amphoteric surfactant, nonionic surfactant, or a combination of two or more thereof. The viscosity control composition may comprise a thickening agent. The viscosity control composition may comprise a liquid carrier.

The first surfactant of the surfactant blend may comprise a biosurfactant. The term “biosurfactant” may refer to a compound generally of biological origin. In some embodiments, the biosurfactant may be produced from fermentation. As such, the biosurfactant may be formed from raw materials that include sugars or sugars and glycerides and/or fatty acids. Biosurfactants do not undergo chemical reaction such as the synthetic glycolipids e.g. alkyl polyglycosides (APG). Biosurfactants can be found in living organisms, or arise in the cultivation of various microorganisms such as e.g., Fungi, yeasts, algae, viruses or bacteria or enzymes.

Biosurfactants may comprise rhamnolipids, glucoselipids, sophorolipids, trehaloselipids, cellobioselipids and mixtures thereof. Biosurfactants may be obtained directly by the use of natural raw materials through microbiological processes, while the production of synthetic glycolipids usually requires further chemical steps, such as the reduction of fatty acid in the fatty alcohol. Due to the biological modification of the sugar part, glycolipid biosurfactants have special properties on, for example, as known in the art, a good fat dissolving power. The structure of the biosurfactants and the chain lengths of the hydrophobic part varies depending on the microorganism or substrate used.

Non-limiting examples of rhamnolipids include compounds having the formula (I):

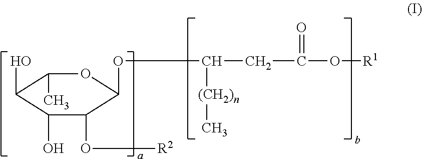

where a is 1 or 2; b is 1 or 2, n is 2 to 24 (whereby this chain may be branched, optionally substituted, in particular hydroxy-substituted, optionally unsaturated, in particular optionally mono-, di- or tri-unsaturated hydrocarbon radical, preferably selected from the group consisting of pentenyl, heptenyl, nonenyl, undecenyl and tridecenyl and (CH₂)ₒ—CH₃ with o=1 to 23, preferably 4 to 12, very particularly preferably o=6); R¹ is H or a cation, preferably H, or a monovalent solubilizing cation, R² is H or the group

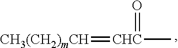

preferably H; m is 4 to 10; and the values of m and n need not be the same at each occurrence.

Rhamnolipids can be produced by bacterial fermentation. This is inherently advantageous in that products of bacterial fermentation can generally be derived from renewable raw materials and are likely to be biodegradable after use. Another advantage of the surfactants of formula (I) is that they can be produced as a by-product of enzyme manufacture.

Rhamnolipids can be produced by bacteria of the genus Pseudomonas. The bacterial fermentation typically utilizes as substrates a sugar or glycerol or an alkane or mixtures thereof.

Any sample of rhamnolipid will generally contain a variety of individual compounds within the general formula (I). The proportions of individual compounds is governed by the microorganism species, and the particular strain employed for fermentation, the substrate materials supplied to the fermentation, and other fermentation conditions.

The bacterial fermentation generally produces compounds in which R¹ is hydrogen or a solubilizing cation. Such compounds can undergo conversion between the salt and the acid forms in aqueous solution, according to the pH of the solution. Common solubilizing cations are alkali metal, ammonium and alkanolamine.

The term “di-rhamnolipid” in connection with the present invention is understood to mean compounds of the general formula (I) or salts thereof in which a=2. The term “mono-rhamnolipid” in connection with the present invention means compounds of the general formula (I) or salts thereof in which a=1.

Non-limiting examples of glucoselipids compounds having formula (II).

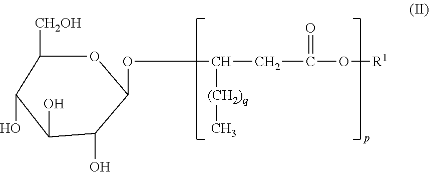

where R¹ is H or a cation; p is 1 to 4; and q is 4 to 10, preferably 6.

Glucoselipids can be produced by the bacterium Alcaligenes Sp.MMl. The glucoselipids are recovered from the fermentation broth via solvent extraction using ethyl ether or a mixture of either dichloromethane:methanol or chloroform:methanol.

Non-limiting examples of sophoroselipids include compounds having the formula (III)

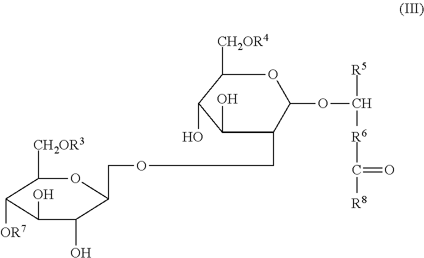

where R³ and R⁴ are individually H or an acetyl group; R⁵ is a saturated or unsaturated, hydroxylated or non-hydroxylated hydrocarbon group having 1 to 9 carbon atoms, preferably being a methyl group; R⁶ is a saturated or unsaturated hydroxylated or non-hydroxylated hydrocarbon group having 1 to 19 carbon atoms; with the proviso that the total number of carbon atoms in the groups R⁵ and R⁶ does not exceed 20 and is preferably from 14 to 18.

The sophoroselipid may be incorporated as either the open chain free acid form, where R⁷ is H and R⁸ is OH, or in its lactone form, where a lactone ring is formed between R⁷ and R⁸ as shown by formula (IV).

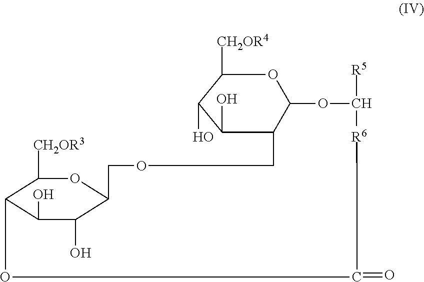

where R³, R⁴ and R⁶ are as defined above; with the proviso that at least one of R³ and R⁴ is an acetyl group.

Sophoroselipids can be produced by yeast cells, for example Torulopsis apicola and Torulopsis bombicola. The fermentation process typically utilizes sugars and alkanes as substrates. The resultant product is a mixture of various open-chain sophorolipids and sophoroselipid lactones, which may be utilized as a mixture, or the required form can be isolated. When the glycolipid biosurfactant comprises sophoroselipids, the weight ratio of sophoroselipids to additional surfactant is preferably in the range 4:1 to 3:2 and is more preferably 4:1.

Non-limiting examples of trehaloselipids include compounds having the general formula (V).

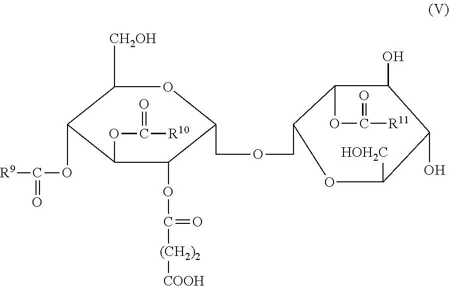

where R⁹, R¹⁰ and R¹¹ are individually a saturated or unsaturated, hydroxylated or non-hydroxylated hydrocarbon of 5 to 13 carbon atoms.

Trehaloselipids can be produced by bacteria fermentation using the marine bacterium Arthrobacter sp. Ek 1 or the fresh water bacterium Rhodococcus erythropolis.

Non-limiting examples of cellobioselipids may include compounds having the general formula (VI).

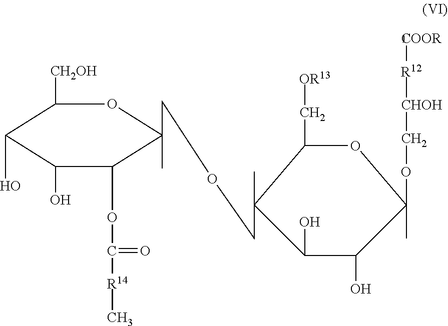

where R¹ is H or a cation; R¹² is a saturated or non-saturated, hydroxylated or non-hydroxylated hydrocarbon having 9 to 15 carbon atoms, preferably 13 carbon atoms; R¹³ is H or an acetyl group; R¹⁴ is a saturated or non-saturated, hydroxylated or non-hydroxylated hydrocarbon having 4 to 16 carbon atoms.

Cellobioselipids can be produced by fungi cells from the genus Ustilago. When the glycolipid biosurfactant comprises cellobioselipids, the weight ratio of cellobioselipids to additional surfactant is preferably in the range 4:1 to 2:3.

The biosurfactant (first surfactant) may be present in an amount ranging from about 0.001 wt. % to about 13.0 wt. %, based on the total weight of the viscosity control composition—including all amounts and sub-ranges there-between. In some embodiments, the biosurfactant (first surfactant) may be present in an amount ranging from about 0.1 wt. % to about 4.0 wt. %, based on the total weight of the viscosity control composition—including all amounts and sub-ranges there-between. In some embodiments, the biosurfactant (first surfactant) may be present in an amount ranging from about 0.1 wt. % to about 3.0 wt. %, based on the total weight of the viscosity control composition—including all amounts and sub-ranges there-between. In some embodiments, the biosurfactant (first surfactant) may be present in an amount ranging from about 0.5 wt. % to about 3.0 wt. %, based on the total weight of the viscosity control composition—including all amounts and sub-ranges there-between. In some embodiments, the biosurfactant (first surfactant) may be present in an amount ranging from about 0.6 wt. % to about 2.5 wt. %, based on the total weight of the viscosity control composition—including all amounts and sub-ranges there-between. In some embodiments, the biosurfactant (first surfactant) may be present in an amount ranging from about 0.7 wt. % to about 2.0 wt. %, based on the total weight of the viscosity control composition—including all amounts and sub-ranges there-between. In some embodiments, the biosurfactant (first surfactant) may be present in an amount ranging from about 0.8 wt. % to about 1.5 wt. %, based on the total weight of the viscosity control composition—including all amounts and sub-ranges there-between. In some embodiments, the biosurfactant (first surfactant) may be present in an amount ranging from about 0.9 wt. % to about 1.2 wt. %, based on the total weight of the viscosity control composition—including all amounts and sub-ranges there-between. In some embodiments, the biosurfactant (first surfactant) may be present in an amount of about 1.0 wt. %, based on the total weight of the viscosity control composition.

The second surfactant of the surfactant blend may comprise an anionic surfactant. The one or more anionic surfactant(s) may be selected from non-sulfate based anionic surfactants, such as sulfonated monoglycerides of fatty acids, isethionates, sarcosinates, taurate, or a combination of two or more thereof. Although the one or more anionic surfactant(s) are typically chosen from non-sulfate based anionic surfactants, the oral care composition may, in some embodiments, include a sulfate based anionic surfactants selected from ammonium lauryl sulfate, ammonium lauryl ether sulfate, sodium dodecyl sulfate, sodium coco-sulfate, ammonium coco-sulfate, and a combination of two or more thereof. For example, in some embodiments, the surfactant system includes a sulfate based anionic surfactant, wherein the oral care composition is substantially free of or free of sodium lauryl sulfate and/or sodium lauryl ether sulfate.

Non-limiting examples of suitable anionic surfactants that may be used are alkyl benzene sulphonates, alkyl ether sulphates, olefin sulphonates, alkyl sulphonates, secondary alkyl sulphonates, fatty acid ester sulphonates, dialkyl sulphosuccinates, and alkyl orthoxylene sulphonates. Specific examples of alkyl benzene sulphonates include alkali metal, ammonium or alkanolamine salts of alkylbenzene sulphonates having from 10 to 18 carbon atoms in the alkyl group.

Suitable alkyl and alkylether sulphates include those having from 10 to 24 carbon atoms in the alkyl group, the alkylether sulphates have from 1 to 5 ethylene oxide groups.

Suitable olefin sulphonates are those prepared by sulphonation of C₁₀-C₂₄ alpha-olefins and subsequent neutralisation and hydrolysis of the sulphonation reaction product.

Specific examples of alkyl sulphates, or sulphated fatty alcohol salts, include those of mixed alkyl chain length, in which the ratio of C₁₂ alkyl chains to C₁₈ alkyl chains is in the range of from 9:4 to 1:6. A suitable material can be obtained from a mixture of synthetic lauryl and oleyl alcohols in appropriate properties.

Specific examples of fatty acid ester sulphonates include those of the general formula

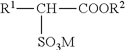

wherein R¹ is derived from tallow, palm or coconut oil and R² is a short chain alkyl group such as butyl.

Specific examples of dialkyl sulphosuccinates include those in which both alkyl substituent contains at least 4 carbon atoms, and together contain 12 to 20 carbon atoms in total, such as di-C₈ alkyl sulphosuccinate.

Specific examples of alkyl orthoxylene sulphonates include those in which the alkyl group contains from 12 to 24 carbon atoms.

Other anionic surfactants which may be used include alkali metal soaps of a fatty acid, preferably one containing 12 to 18 carbon atoms. Typical such acids are oleic acid, ricinoleic acid and fatty acids derived from castor oil, rapeseed oil, groundnut oil, coconut oil, palmkernal oil or mixtures thereof. The sodium or potassium soaps of these acids can be used. As well as fulfilling the role of surfactants, soaps can act as detergency builders or fabric conditioners.

Dialkyl sulphosuccinates are of especial interest as lamellar phase anionic surfactants for use in the present invention.

Suitable anionic surfactants are preferably C₈-C₁₈-alkylbenzenesulfonates, in particular having about 12 C atoms in the alkyl moiety, C₈-C₂₀-alkanesulfonates, C₈-C₁₈-monoalkyl sulfates (fatty alcohol sulfates), C₈-C₁₈-alkyl polyglycol ether sulfates with 2 to 6 ethylene oxide units (EO) in the ether portion and sulfosuccinic mono- and di-C₈-C₁₈ alkyl esters. Furthermore, C₈-C₁₈-α-olefinsulfonates, sulfonated C₈-C₁₈-fatty acids, in particular dodecylbenzenesulfonate, C₈-C₂₂-Carbonsaureamidethersulfate, C₈-C₁₈-Alkylpolyglykolethercarboxylate, C₈-C₁₈—N-Acyltauride, N-Acylaminosaurederivate as N-acyl aspartates or N-acyl glutamates, C₈-C₁₈—N sarcosinates and C₈-C₁₈-Alkylisethionate or mixtures thereof.

In a non-limiting embodiment, the anionic surfactant is an amine containing compound. In a non-limiting embodiment, the anionic surfactant is a C₈-C₁₈—N sarcosinate containing compound. In some embodiments, the anionic surfactant is sodium lauroyl sarcosinate. In some embodiments, the anionic surfactant comprises an amino acid based anionic surfactant selected from acyl glutamates and glycinates.

The anionic surfactants are preferably used as sodium salts, but may also be present as other alkali or alkaline earth metal salts, for example magnesium salts, and in the form of ammonium or mono-, di-, tri- or tetraalkylammonium salts, in the case of the sulfonates also in the form their corresponding acid, e.g., Dodecylbenzenesulfonic.

Examples of such surfactants are sodium cocoalkyl sulfate, sodium sec-alkanesulfonate having about 15 carbon atoms and sodium dioctylsulfosuccinate. Sodium fatty alkyl sulfates and fatty alkyl+2EO ether sulfates having 12 to 14 C atoms have proven to be particularly suitable.

C₈-C₁₈-alcohol polyglycol ethers, i.e., ethoxylated and/or propoxylated alcohols having 8 to 18 C atoms in the alkyl moiety and 2 to 15 ethylene oxide (EO) and/or propylene oxide units (PO), C₈—are noteworthy nonionic surfactants. C₁₈-carboxylic acid polyglycol esters having 2 to 15 EO, for example tallow fatty acid+6-EO esters, ethoxylated fatty acid amides having 12 to 18 C atoms in the fatty acid part and 2 to 8 EO, long-chain amine oxides having 14 to 20 C atoms and long-chain alkylpolyglycosides to mention with 8 to 14 carbon atoms in the alkyl moiety and 1 to 3 glycoside units. Examples of such surfactants are oleyl-cetyl-alcohol with 5 EO, nonylphenol with 10 EO, lauric acid diethanolamide, Kokosalkyldimethylaminoxid and Kokosalkylpolyglucosid with an average of 1.4 glucose units. Particular preference is given to using fatty alcohol polyglycol ethers having in particular 2 to 8 EO, for example C₁₂-14-fatty alcohol+4-EO ether, amine oxides and C₈₋₁₀-alkylpolyglucosides having 1 to 2 glycoside units.

The anionic (e.g., second surfactant) may be present in an amount ranging from about 0.1 wt. % to about 13.0 wt. %, based on the total weight of the viscosity control composition—including all amounts and sub-ranges there-between. In some embodiments, the anionic (second surfactant) may be present in an amount ranging from about 0.3 wt. % to about 6.0 wt. %, based on the total weight of the viscosity control composition—including all amounts and sub-ranges there-between. In some embodiments, the anionic (second surfactant) may be present in an amount ranging from about 0.4 wt. % to about 5.0 wt. %, based on the total weight of the viscosity control composition—including all amounts and sub-ranges there-between. In some embodiments, the anionic (second surfactant) may be present in an amount ranging from about 0.5 wt. % to about 3.5 wt. %, based on the total weight of the viscosity control composition—including all amounts and sub-ranges there-between. In some embodiments, the anionic (second surfactant) may be present in an amount ranging from about 0.7 wt. % to about 3.0 wt. %, based on the total weight of the viscosity control composition—including all amounts and sub-ranges there-between. In some embodiments, the anionic (second surfactant) may be present in an amount ranging from about 0.9 wt. % to about 2.5 wt. %, based on the total weight of the viscosity control composition
including all amounts and sub-ranges there-between. In some embodiments, the anionic (second surfactant) may be present in an amount ranging from about 1.1 wt. % to about 2.0 wt. %, based on the total weight of the viscosity control composition—including all amounts and sub-ranges there-between. In some embodiments, the anionic (e.g., second surfactant) may be present in an amount ranging from about 1.3 wt. % to about 1.7 wt. %, based on the total weight of the viscosity control composition—including all amounts and sub-ranges there-between. In some embodiments, the anionic (second surfactant) may be present in an amount of about 1.5 wt. %, based on the total weight of the viscosity control composition.

The third surfactant of the surfactant blend may comprise an amphoteric compound, a zwitterionic compound, and/or combinations thereof. Suitable amphoteric or zwitterionic surfactants for use in the composition herein include those which are known for use in hair care or other personal care cleansing.

Amphoteric surfactants suitable for use in the composition are well known in the art, and include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Suitable examples of such amphoteric surfactants include, but are not limited to, sodium cocaminopropionate, sodium cocaminodipropionate, sodium cocoamphoacetate, sodium cocoamphohydroxypropylsulfonate, sodium cocoamphopropionate, sodium cornamphopropionate, sodium lauraminopropionate, sodium lauroamphoacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphohydroxypropylsulfonate, ammonium cocoamphopropionate, ammonium cornamphopropionate, ammonium lauraminopropionate, ammonium lauroamphoacetate, ammonium lauroamphohydroxypropylsulfonate, ammonium lauroamphopropionate, ammonium cornamphopropionate, ammonium lauriminodipropionate, triethanonlamine cocaminopropionate, triethanonlamine cocaminodipropionate, triethanonlamine cocoamphoacetate, triethanonlamine cocoamphohydroxypropylsulfonate, triethanonlamine cocoamphopropionate, triethanonlamine cornamphopropionate, triethanonlamine lauraminopropionate, triethanonlamine lauroamphoacetate, triethanonlamine lauroamphohydroxypropylsulfonate, triethanonlamine lauroamphopropionate, triethanonlamine cornamphopropionate, triethanonlamine lauriminodipropionate, cocoamphodipropionic acid, disodium caproamphodiacetate, disodium caproamphoadipropionate, disodium capryloamphodiacetate, disodium capryloamphodipriopionate, disodium cocoamphocarboxyethylhydroxypropylsulfonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethy-7 carboxyamphodiacetate, lauraminopropionic acid, lauroamphodipropionic acid, lauryl aminopropylglycine, lauryl diethylenediaminoglycine, and combinations thereof.

In one embodiment, the amphoteric surfactant is a surfactant according to the following structure:

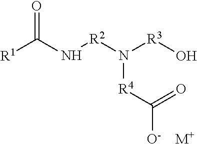

wherein R¹ is a C-linked monovalent substituent selected from the group consisting of substituted alkyl systems comprising 9 to 15 carbon atoms, unsubstituted alkyl systems comprising 9 to 15 carbon atoms, straight alkyl systems comprising 9 to 15 carbon atoms, branched alkyl systems comprising 9 to 15 carbon atoms, and unsaturated alkyl systems comprising 9 to 15 carbon atoms; R², R³, and R⁴ are each independently selected from the group consisting of C-linked divalent straight alkyl systems comprising 1 to 3 carbon atoms, and C-linked divalent branched alkyl systems comprising 1 to 3 carbon atoms; and M+ is a monovalent counter-ion selected from the group consisting of sodium, ammonium and protonated triethanolamine.

Specific examples of suitable surfactants include sodium cocoamphoacetate, sodium cocoamphodiacetate, sodium lauroamphoacetate, sodium lauroamphodiacetate, ammonium lauroamphoacetate, ammonium cocoamphoacetate, triethanolamine lauroamphoacetate, and triethanolamine cocoamphoacetate.

Zwitterionic surfactants suitable for use in the composition are well known in the art, and include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. Suitable zwitterionic surfactants include, but are not limited to, cocamidoethyl betaine, cocamidopropylamine oxide, cocamidopropyl betaine, cocamidopropyl dimethylaminohydroxypropyl hydrolyzed collagen, cocamidopropyldimonium hydroxypropyl hydrolyzed collagen, cocamidopropyl hydroxysultaine, cocobetaineamido amphopropionate, coco-betaine, coco-hydroxysultaine, coco/oleamidopropyl betaine, coco-sultaine, lauramidopropyl betaine, lauryl betaine, lauryl hydroxysultaine, lauryl sultaine, and combinations thereof.

In some embodiments, the surfactant blend comprises amphoacetate containing compound (e.g., a second or third surfactant). In some embodiments, the surfactant blend comprises a lauroamphoacetate compound. In some embodiments, the surfactant blend comprises (e.g., a third surfactant) sodium lauroamphoacetate.

The zwitterionic/amphoteric surfactant (e.g., third surfactant) may be present in an amount ranging from about 1.0 wt. % to about 24.0 wt. %, based on the total weight of the viscosity control composition—including all amounts and sub-ranges there-between. In some embodiments, the zwitterionic/amphoteric surfactant (e.g., third surfactant) may be present in an amount ranging from about 2.0 wt. % to about 14.0 wt. %, based on the total weight of the viscosity control composition—including all amounts and sub-ranges there-between. In some embodiments, the zwitterionic/amphoteric surfactant (e.g., third surfactant) may be present in an amount ranging from about 3.0 wt. % to about 12.0 wt. %, based on the total weight of the viscosity control composition—including all amounts and sub-ranges there-between. In some embodiments, the zwitterionic/amphoteric surfactant (e.g., third surfactant) may be present in an amount ranging from about 4.0 wt. % to about 11.0 wt. %, based on the total weight of the viscosity control composition—including all amounts and sub-ranges there-between. In some embodiments, the zwitterionic/amphoteric surfactant (e.g., third surfactant) may be present in an amount ranging from about 5.0 wt. % to about 10.0 wt. %, based on the total weight of the viscosity control composition—including all amounts and sub-ranges there-between. In some embodiments, the zwitterionic/amphoteric surfactant (e.g., third surfactant) may be present in an amount ranging from about 6.0 wt. % to about 9.0 wt. %, based on the total weight of the viscosity control composition—including all amounts and sub-ranges there-between. In some embodiments, the zwitterionic/amphoteric surfactant (e.g., third surfactant) may be present in an amount ranging from about 6.5 wt. % to about 8.0 wt. %, based on the total weight of the viscosity control composition—including all amounts and sub-ranges there-between. In some embodiments, the zwitterionic/amphoteric surfactant (e.g., third surfactant) may be present in an amount ranging from about 7.0 wt. % to about 8.0 wt. %, based on the total weight of the viscosity control composition—including all amounts and sub-ranges there-between. In some embodiments, the zwitterionic/amphoteric surfactant (e.g., third surfactant) may be present in an amount of about 7.5 wt. %, based on the total weight of the viscosity control composition.

The surfactant blend may be present in an amount ranging from about 2 wt. % to about 25 wt. %, based on the total weight of the viscosity control composition—including all wt. % and sub-ranges-there-between. In some embodiments, the surfactant blend may be present in an amount ranging from about 3 wt. % to about 18 wt. %—based on the total weight of the viscosity control composition—including all wt. % and sub-ranges-there-between. In some embodiments, the surfactant blend may be present in an amount ranging from about 4 wt. % to about 17 wt. %—based on the total weight of the viscosity control composition—including all wt. % and sub-ranges-there-between. In some embodiments, the surfactant blend may be present in an amount ranging from about 5 wt. % to about 16 wt. %—based on the total weight of the viscosity control composition—including all wt. % and sub-ranges-there-between. In some embodiments, the surfactant blend may be present in an amount ranging from about 6 wt. % to about 15 wt. %—based on the total weight of the viscosity control composition—including all wt. % and sub-ranges-there-between. In some embodiments, the surfactant blend may be present in an amount ranging from about 7 wt. % to about 14 wt. %—based on the total weight of the viscosity control composition—including all wt. % and sub-ranges-there-between. In some embodiments, the surfactant blend may be present in an amount ranging from about 8 wt. % to about 13 wt. %—based on the total weight of the viscosity control composition—including all wt. % and sub-ranges-there-between. In some embodiments, the surfactant blend may be present in an amount ranging from about 8 wt. % to about 12 wt. %—based on the total weight of the viscosity control composition—including all wt. % and sub-ranges-there-between. In some embodiments, the surfactant blend may be present in an amount ranging from about 9 wt. % to about 11 wt. %—based on the total weight of the viscosity control composition—including all wt. % and sub-ranges-there-between. In some embodiments, the surfactant blend may be present in an amount of about 10 wt. % based on the total weight of the viscosity control.

The anionic surfactant (e.g., second surfactant) and the biosurfactant (e.g., first surfactant) may be present in a weight ratio ranging from about 1:20 to about 20:1—including all ratios sub-ranges there-between. In some embodiments, the anionic surfactant (e.g., second surfactant) and the biosurfactant (e.g., first surfactant) may be present in a weight ratio ranging from about 0.7:1 to about 2.0:1—including all ratios sub-ranges there-between. In some embodiments, the anionic surfactant (e.g., second surfactant) and the biosurfactant (e.g., first surfactant) may be present in a weight ratio ranging from about 0.8:1 to about 1.9:1—including all ratios sub-ranges there-between. In some embodiments, the anionic surfactant (e.g., second surfactant) and the biosurfactant (e.g., first surfactant) may be present in a weight ratio ranging from about 1.0:1 to about 1.8:1—including all ratios sub-ranges there-between. In some embodiments, the anionic surfactant (e.g., second surfactant) and the biosurfactant (e.g., first surfactant) may be present in a weight ratio ranging from about 1.2:1 to about 1.7:1—including all ratios sub-ranges there-between. In some embodiments, the anionic surfactant (e.g., second surfactant) and the biosurfactant (e.g., first surfactant) may be present in a weight ratio ranging from about 1.4:1 to about 1.6:1—including all ratios sub-ranges there-between. In some embodiments, the anionic surfactant (e.g., second surfactant) and the biosurfactant (e.g., first surfactant) may be present in a weight ratio of about 1.5:1.

The amphoteric/zwitterionic surfactant (e.g., third surfactant) and the biosurfactant (e.g., first surfactant) may be present in a weight ratio ranging from about 1:10 to about 10:1—including all ratios and sub-ranges there-between. In some embodiments, the amphoteric/zwitterionic surfactant (e.g., third surfactant) and the biosurfactant (e.g., first surfactant) may be present in a weight ratio ranging from about 5.5:1 to about 9.5:1—including all ratios and sub-ranges there-between. In some embodiments, the amphoteric/zwitterionic surfactant e.g., (third surfactant) and the biosurfactant (e.g., first surfactant) may be present in a weight ratio ranging from about 6:1 to about 9:1—including all ratios and sub-ranges there-between. In some embodiments, the amphoteric/zwitterionic surfactant (e.g., third surfactant) and the biosurfactant (e.g., first surfactant) may be present in a weight ratio ranging from about 6.5:1 to about 8.5:1—including all ratios and sub-ranges there-between. In some embodiments, the amphoteric/zwitterionic surfactant (e.g., third surfactant) and the biosurfactant (e.g., first surfactant) may be present in a weight ratio ranging from about 7:1 to about 8:1—including all ratios and sub-ranges there-between. In some embodiments, the amphoteric/zwitterionic surfactant (e.g., third surfactant) and the biosurfactant (e.g., first surfactant) may be present in a weight ratio of about 7.5:1.

The amphoteric/zwitterionic surfactant (e.g., third surfactant) and the anionic surfactant (e.g., second surfactant) may be present in a weight ratio ranging from about 1:13 to about 13:1—including all ratios and sub-ranges there-between. In some embodiments, the amphoteric/zwitterionic surfactant (e.g., third surfactant) and the anionic surfactant (e.g., second surfactant) may be present in a weight ratio ranging from about 3:1 to about 7:1—including all ratios and sub-ranges there-between. In some embodiments, the amphoteric/zwitterionic surfactant (e.g., third surfactant) and the anionic surfactant (e.g., second surfactant) may be present in a weight ratio ranging from about 3.5:1 to about 6.5:1—including all ratios and sub-ranges there-between. In some embodiments, the amphoteric/zwitterionic surfactant (e.g., third surfactant) and the anionic surfactant (e.g., second surfactant) may be present in a weight ratio ranging from about 4:1 to about 6:1—including all ratios and sub-ranges there-between. In some embodiments, the amphoteric/zwitterionic surfactant (e.g., third surfactant) and the anionic surfactant (e.g., second surfactant) may be present in a weight ratio ranging from about 4.5:1 to about 5.5:1—including all ratios and sub-ranges there-between. In some embodiments, the amphoteric/zwitterionic surfactant (e.g., third surfactant) and the anionic surfactant (e.g., second surfactant) may be present in a weight ratio of about 5:1.

The viscosity control composition may comprise a thickening agent. The thickening agent may be selected from one or more polysaccharide. In some embodiments, the polysaccharide may comprise one or more of xanthan gum, tragacanth gum, carrageenan gum (also referred as just “carrageenan”), alginates, agar-agar, gum Arabic, guar gum, gellan gum, pectin, cellulose, welan, dituan gum, locust bean gum extract seeds of the carob tree, dammar gum, kauri gum, spruce gum, gum from Fenugreek, gum anima (western or eastern), or a combination of two or more thereof. In some embodiments, the viscosity control composition has one or more thickening agents consisting of polysaccharide(s).

In further embodiments, the viscosity control composition has a reduced amount, is essentially free of, or free of thickening agents selected from acrylates, acrylamides, colloidal silicates or a combination of all of the foregoing. For example, the viscosity control composition may have acrylates, acrylamides, colloidal silicates, individually, in an amount of about 3 wt. % or less, about 2 wt. % or less, about 1 wt. % or less, about 0.5 wt. % or less, about 0.1 wt. % or less, about 0.05 wt. % or less, or about 0.01 wt. % or less, based on the total weight of the viscosity control composition. In at least one embodiment, the viscosity control composition is essentially free of or free of acrylates, acrylamides, and/or colloidal silicates.

In a preferred embodiment, the thickening agent is carrageenan. It has been surprisingly discovered that using a thickening agent of carrageenan in combination with the surfactant blend of the present invention results in an unexpected synergy resulting in an effective tool for building viscosity. Specifically, the addition of carrageenan to the blend of biosurfactant, anionic surfactant, and zwitterionic/amphoteric surfactant provides a surprising increased in viscosity of the overall blend—as compared to other thickening agents (e.g., carboxyl methylcellulose).

Specifically, the thickening agent—specifically, carrageenan—may be added to the viscosity control composition in a quasi-linear manner to build overall viscosity in a non-linear manner (i.e., exceeding linear increases in viscosity based on linear additions of the carrageenan to the overall viscosity control composition). As such the newly discovered synergy of the surfactant blend with the carrageenan provides an efficient method of increasing viscosity without requiring large amounts of thickening agent.

The thickening agent may be present in an amount ranging from about 0.05 wt. % to about 10 wt. %—based on the total weight of the viscosity control composition—including all wt. % and sub-ranges-there-between. In some embodiments, the thickening agent may be present in an amount ranging from about 0.1 wt. % to about 5.0 wt. %—based on the total weight of the viscosity control composition—including all wt. % and sub-ranges-there-between. In some embodiments, the thickening agent may be present in an amount ranging from about 0.2 wt. % to about 4.0 wt. %—based on the total weight of the viscosity control composition—including all wt. % and sub-ranges-there-between. In some embodiments, the thickening agent may be present in an amount ranging from about 0.2 wt. % to about 3.0 wt. %—based on the total weight of the viscosity control composition—including all wt. % and sub-ranges-there-between. In some embodiments, the thickening agent may be present in an amount ranging from about 0.2 wt. % to about 2.0 wt. %—based on the total weight of the viscosity control composition—including all wt. % and sub-ranges-there-between.

In some embodiments, the thickening agent may be present in an amount of about 0.1 wt. %—based on the total weight of the viscosity control composition. In some embodiments, the thickening agent may be present in an amount of about 0.2 wt. %—based on the total weight of the viscosity control composition. In some embodiments, the thickening agent may be present in an amount of about 0.3 wt. %—based on the total weight of the viscosity control composition. In some embodiments, the thickening agent may be present in an amount of about 0.4 wt. %—based on the total weight of the viscosity control composition. In some embodiments, the thickening agent may be present in an amount of about 0.5 wt. %—based on the total weight of the viscosity control composition. In some embodiments, the thickening agent may be present in an amount of about 0.6 wt. %—based on the total weight of the viscosity control composition. In some embodiments, the thickening agent may be present in an amount of about 0.7 wt. %—based on the total weight of the viscosity control composition. In some embodiments, the thickening agent may be present in an amount of about 0.8 wt. %—based on the total weight of the viscosity control composition. In some embodiments, the thickening agent may be present in an amount of about 0.9 wt. %—based on the total weight of the viscosity control composition. In some embodiments, the thickening agent may be present in an amount of about 1.0 wt. %—based on the total weight of the viscosity control composition. In some embodiments, the thickening agent may be present in an amount of about 1.1 wt. %—based on the total weight of the viscosity control composition. In some embodiments, the thickening agent may be present in an amount of about 1.2 wt. %—based on the total weight of the viscosity control composition. In some embodiments, the thickening agent may be present in an amount of about 1.3 wt. %—based on the total weight of the viscosity control composition. In some embodiments, the thickening agent may be present in an amount of about 1.4 wt. %—based on the total weight of the viscosity control composition. In some embodiments, the thickening agent may be present in an amount of about 1.5 wt. %—based on the total weight of the viscosity control composition. In some embodiments, the thickening agent may be present in an amount of about 1.6 wt. %—based on the total weight of the viscosity control composition. In some embodiments, the thickening agent may be present in an amount of about 1.7 wt. %—based on the total weight of the viscosity control composition. In some embodiments, the thickening agent may be present in an amount of about 1.8 wt. %—based on the total weight of the viscosity control composition. In some embodiments, the thickening agent may be present in an amount of about 1.9 wt. %—based on the total weight of the viscosity control composition. In some embodiments, the thickening agent may be present in an amount of about 2.0 wt. %—based on the total weight of the viscosity control composition. In some embodiments, the thickening agent may be present in an amount of about 2.1 wt. %—based on the total weight of the viscosity control composition.

The viscosity control composition may comprise a liquid carrier. The liquid carrier may comprise one or more of a solvent, diluent, and combinations thereof. The liquid carrier may comprise one or more of water, alcohols (e.g., ethanol, isopropanol, propanol, or mixtures thereof), and mixtures thereof. In at least one embodiment, the carrier comprises water.

In some embodiments, the carrier comprises or excludes one polyol selected from glycerin, ethylene glycol, propylene glycol, butylene glycol, hexylene glycol, diglycerin, diethylene glycol, dipropylene glycol, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and a mixture of two or more thereof.

The amount of carrier present in the personal care formulation may vary, e.g., from about 75 to about 98 wt. %, based on the total weight of the viscosity control composition—including all wt. % and sub-ranges there-between. In some embodiments, the amount of carrier present in the personal care formulation may vary, e.g., from about 81 to about 93 wt. %, based on the total weight of the viscosity control composition—including all wt. % and sub-ranges there-between. In some embodiments, the amount of carrier present in the personal care formulation may vary, e.g., from about 85 to about 91 wt. %, based on the total weight of the viscosity control composition—including all wt. % and sub-ranges there-between. In some embodiments, the amount of carrier present in the personal care formulation may vary, e.g., from about 88 to about 90 wt. %, based on the total weight of the viscosity control composition—including all wt. % and sub-ranges there-between.

In some embodiments, the surfactant blend, the thickening agent, and the liquid carrier total to about 100 wt. % of the viscosity control composition.

In a preferred embodiment, the viscosity control composition may comprise a surfactant blend comprising a biosurfactant that includes rhamnolipid, an anionic surfactant that includes sodium lauroyl sarcosinate, and a zwitterionic surfactant that includes sodium lauroamphoacetate, whereby the viscosity control composition further comprises a thickening agent that is a gum that includes carrageenan as well as liquid carrier.

In such embodiment, the rhamnolipid may be present in an amount of about 1.0 wt. % based on the total weight of the viscosity control composition; the sodium lauroyl sarcosinate is present in an amount of about 1.5 wt. % based on the total weight of the viscosity control composition; the sodium lauroamphoacetate is present in an amount of about 7.5 wt. % based on the total weight of the viscosity control composition; the carrageenan is present in an amount ranging from about 0.2 wt. % to about 2.0 wt. % based on the total weight of the viscosity control agent—whereby the rhamnolipid, sodium lauroyl sarcosinate, sodium lauroamphoacetate, carrageenan, and liquid carrier total to 100 wt. % of the viscosity control composition.

The present invention further includes a personal care composition that comprises the viscosity control composition. The personal care composition may be cleansing product. Non-limiting examples of the personal care composition may be a liquid hand soap, facial cleanser, shower gel, shampoo, conditioner, body wash, or skin cleanser.

In addition to the viscosity control composition, the personal care composition may further comprise one or more personal care ingredients. Further non-limiting examples of such personal care ingredients include preservatives, anti-bacterial agents, perfumes or fragrances, coloring agents or dyes, conditioning agents, hair bleaching agents, moisturizers, emollients, pharmaceutical actives, vitamins or nutrients, sunscreens, deodorants, sensates, plant extracts, nutrients, astringents, cosmetic particles, absorbent particles, adhesive particles, hair fixatives, fibers, reactive agents, skin lightening agents, skin tanning agents, anti-dandruff agents, perfumes, exfoliating agents, acids, bases, humectants, enzymes, suspending agents, pH modifiers, hair colorants, hair perming agents, pigment particles, anti-acne agents, anti-microbial agents, sunscreens, tanning agents, exfoliation particles, hair growth or restorer agents, insect repellents, shaving lotion agents, co-solvents or other additional solvents, and similar other materials.

In some embodiments, the personal care ingredient may comprise at least one perfume. The perfume may contain one or more essential oils. The at least one essential oil may be selected in a non-limiting fashion from the following essential oils: ambrette, angelica, anise, amyris, bay, bergamot, basil, bois-de-rose, cade, cajiput, camphor, cananga, java cassia, clary sage, curry, calamus, costus, carrot, cedar wood, cedar, cinnamon, citronella java, clove, cypress, cyperiol, chamomile blue, chamomile roman, davana, dill, elemi, eucalyptus, eucalyptus globules, frank incense, geranium, ginger grass, ginger lily, galangal, gurjam, grape fruit, jasminum, juniper berry, juniper, kapoor katcheri, lavender, lemon, lemon grass, lemon melissa, lime, laur spearmint, lemon balm, Litsea cubeba, Mentha citrata, Mentha piperata, Mentha shivalik, mandarin, marjoram, mint, myrtle, nar kachur, neroli, niaouli, orange, sweet orange, oregano, bitter patchouli, petit grain, peppermint, pine, palma rosa, pimento berry, rose wood, rose marry, rosemary, sandal wood, spearmint, sugandh mantri (gandhi roots), spike nard, tarragon, tangerine, tea tree, thyme, thuja wood, tomar (zanthozylum), tagettues, vetiver, valerian, winter green, worm wood (gaultheria fragrantissim wall), ylang, zadoeria and any combination thereof.

In addition, or alternatively to the aforementioned essential oil, the formulation may comprise at least one additional plant extract selected from wasabi, black currant, aspen bark, Lonicera species, and others. In some embodiments, the amount of the perfume is in the range of about 0.1 wt. % to about 5 wt. %, based on the total weight of the personal care composition.

The personal care ingredient may further comprise at least one natural preservative, which may be one or more of an extract of anise, black currant, cinnamon or cinnamon oil, geranium or geranium oil, ginger or ginger oil, Indian ginseng root, lavender, lemongrass, Magnolia acnibio, maritime pine, Mentha piperita, olive leaf, oregano, peppermint, elderberry, rosemary, tea tree, thyme and grapefruit. In some embodiments, the amount of the at least one preservative is in the range of about 0.1 wt. % to about 5 wt. % based on the total weight of the personal care composition.

The personal care composition may also comprise a natural extract, e.g., a plant extract from the one or more of the genus Lonicera, Populus, Salix and Wasabia or combination thereof. It should be understood that the extract may be an extract of more than one plant selected from Lonicera, Populus, Salix and Wasabia and that each plant may be selected from the same genus or from a different genus. It should be further that the present invention further contemplates the personal care composition comprising mixtures of extract, whether prepared and formulated individually or prepared in one-pot from a mixture of plant sources (plant parts).

Non-limiting examples of enzymes may include proteases, lipases, amylases, hydrolases and/or cellulases. In order to protect an enzyme contained in an agent according to the invention from damage such as inactivation, denaturation or decomposition, for example by physical influences, oxidation or proteolytic cleavage, enzyme stabilizers can be added to the enzyme-containing agent.

Depending on the type of enzyme used, suitable enzyme stabilizers are, for example: benzamidine hydrochloride, borax, boric acids, boronic acids or their salts or esters, especially derivatives with aromatic groups, for example substituted phenylboronic acids or their salts or esters; Peptide aldehydes, amino alcohols such as mono-, di-, triethanol- and -propanolamine and mixtures thereof, aliphatic carboxylic acids up to C₁₂, such as succinic acid, other dicarboxylic acids or salts of said acids; end-capped fatty acid amide alkoxylates; lower aliphatic alcohols and especially polyols, for example glycerol, ethylene glycol, propylene glycol or sorbitol; and reducing agents and antioxidants such as sodium sulfite and reducing sugars. Other suitable stabilizers are known in the art.

Particularly suitable according to the invention are biotechnologically produced enzymes with the aid of non-genetically modified organisms (non GMO), stabilizers based on renewable raw materials and/or mineral substances, for example boric acid and/or borax, reducing sugars, succinic acid or other dicarboxylic acids, polyamino compounds in particular based on natural amino acids.

Surprisingly, in the enzyme-free embodiment, an outstanding cleaning performance with respect to carbohydrates is found, which is quite comparable with enzyme-containing agents in the market, this is disclosed by way of example in the exemplary embodiments. A particularly preferred embodiment of the invention is therefore those without cellulases or amylases, very particularly preferred is the enzyme-free embodiment. This is particularly beneficial for consumers with allergies and/or sensitive skin. Enzyme-free embodiments with comparable cleaning power are disclosed herein, e.g., in the embodiments.

Non-limiting examples of anti-bacterial agents may include alcohols, aldehydes, antimicrobial acids or their salts, carboxylic esters, acid amides, phenols, phenol derivatives, diphenyls, diphenylalkanes, urea derivatives, oxygen, nitrogen acetals and formals, benzamidines, isothiazoles and their derivatives are suitable according to the invention Derivatives such as isothiazolines and isothiazolinones, phthalimide derivatives, pyridine derivatives, antimicrobial surface active compounds, guanidines, antimicrobial amphoteric compounds, quinolines, 1,2-dibromo-2,4-dicyanobutane, iodo-2-propynyl-butyl-carbamate, iodine, iodophores and peroxides. Preferred antimicrobial agents are preferably selected from the group comprising ethanol, n-propanol, i-propanol, 1,3-butanediol, phenoxyethanol, 1,2-propylene glycol, glycerol, undecylenic acid, citric acid, lactic acid, benzoic acid, salicylic acid, thymol, 2-Benzyl 4-chlorophenol, 2,2′-methylenebis (6-bromo-4-chlorophenol), 2,4,4′-trichloro-2′-hydroxydiphenyl ether, N-(4-chlorophenyl)-N-(3,4-dichlorophenyl) urea, N, N′-(1,10-decanediyldi-1-pyridinyl-4-ylidene) bis (1-octanamine) dihydrochloride, N, N′-bis (4-Chlorophenyl)-3,12-diimino-2,4,11,13-tetraazatetradecandiimidamide, antimicrobial quaternary surface active compounds, guanidines. Preferred antimicrobial surface active quaternary compounds contain an ammonium, sulfonium, phosphonium, iodonium or arsonium group.

Furthermore, antimicrobial effective essential oils can be used, which at the same time provide for a scenting of the cleaning agent. However, particularly preferred antimicrobial agents are selected from the group comprising salicylic acid, quaternary surfactants, in particular benzalkonium chloride, peroxo compounds, in particular hydrogen peroxide, alkali metal hypochlorite and mixtures thereof.

The personal care composition may be provided in a container—such a re-sealable bottle configured for the personal care composition to be dispensed incrementally at the time of the desired use—e.g., during bathing.

The present further provides a method of manufacturing a personal care product having viscosity control. The method comprising: blending together the viscosity control composition with personal care ingredient to form the personal care composition. Subsequently, the method comprises adding a predetermine volume of the personal care composition to the container to form the personal care product.

### EXAMPLES

The following experiments tested the impact on rheology based on the surfactant blend with various gums on the viscosity control composition.

### Experiment 1—Comparison of Various Gums

The surfactant blend included rhamnolipid, sodium lauroyl sarcosinate (“LS”), and sodium lauromamphoacetate (“LA”). The various gums included carrageenan, carboxymethylcellulose (“CMC”), and xanthan gum (“XG”). The formulation of each example is set forth below in Table 1—each numerical value represents a weight percentage (wt. %).

An experiment was performed to test the impact of malodor reduction over time using the deodorant formulation of the present invention—formulations are set forth below in Table 1.

TABLE 1

| | Comp. | | Comp. | | Comp. | | | |
|---|---|---|---|---|---|---|---|---|
| | Ex. 1 | | Ex. 2 | | Ex. 3 | | Ex. 1 | |
| Rhamnolipid | 1.0 | wt. % | 1.0 | wt. % | 1.0 | wt. % | 1.0 | wt. % |
| LS | 1.5 | wt. % | 1.5 | wt. % | 1.5 | wt. % | 1.5 | wt. % |
| LA | 7.5 | wt. % | 7.5 | wt. % | 7.5 | wt. % | 7.5 | wt. % |
| Carrageenan | — | | — | | — | | 1.0 | wt. % |
| CMC | — | | 1.0 | wt. % | — | | — | |
| XG | — | | — | | 1.0 | wt. % | — | |
| Water | q.s. | | q.s. | | q.s. | | q.s. | |
| Total | 100 | wt. % | 100 | wt. % | 100 | wt. % | 100 | wt. % |
| Viscosity | 723 | cP | 937 | cP | 2278 | cP | 3317 | cP |

Comparative Example 1 (Comp. Ex. 1) omitted each of the carrageenan, CMC, and XG gum and exhibited a viscosity of 723 cP—which serves as a control basis for the other viscosities of Comp. Ex. 2-3 and Ex. 1 to be compared from. The formulation of Comp. Ex. 2 exhibit a minimal increase in viscosity compared to that of Comp. Ex. 1 thus resulting in the build in viscosity due to the presence of the CMC is marginal. The formulation of Comp. Ex. 3 exhibit an increase in viscosity that was more than double compared to that of Comp. Ex. 1, but surprisingly the formulation of Example 1 (Ex. 1) exhibit an unexpected increase in viscosity that is almost five times the viscosity of Comp. Ex. 1 thus resulting in the build in viscosity due to the carrageenan being substantial.

### Experiment 2—Comparison of Carrageenan Loading Levels

TABLE 2

| | Ex. 1 | | Ex. 2 | | Ex. 3 | | Ex. 4 | | Ex. 5 | | Ex. 6 | |
|---|---|---|---|---|---|---|---|---|---|---|---|---|
| Rhamnolipid | 1.0 | wt. % | 1.0 | wt. % | 1.0 | wt. % | 1.0 | wt. % | 1.0 | wt. % | 1.0 | wt. % |
| LS | 1.5 | wt. % | 1.5 | wt. % | 1.5 | wt. % | 1.5 | wt. % | 1.5 | wt. % | 1.5 | wt. % |
| LA | 7.5 | wt. % | 7.5 | wt. % | 7.5 | wt. % | 7.5 | wt. % | 7.5 | wt. % | 7.5 | wt. % |
| Carrageenan | 1.0 | wt. % | 0.2 | wt. % | 0.5 | wt. % | 0.8 | wt. % | 1.5 | wt. % | 2.0 | wt. % |
| Water | q.s. | | q.s. | | q.s. | | q.s. | | q.s. | | q.s. | |
| Total | 100 | wt. % | 100 | wt. % | 100 | wt. % | 100 | wt. % | 100 | wt. % | 100 | wt. % |
| Viscosity | 3317 | cP | 723 | cP | 1427 | cP | 2263 | cP | 6142 | cP | 12080 | cP |

As demonstrated by Table 2, the addition of Carrageenan provides an effective tool for controlling the viscosity build of the surfactant formulation. Specifically, the same viscosity build as Comp. Ex. 1 can be achieved using only ⅕th the amount of gum when that gum is Carrageenan. Additionally, the data of Table 2 demonstrated that Carrageenan imparts a synergistic effect in the build of viscosity as the increasing amount of Carrageenan has a non-linear build in viscosity of the resulting surfactant formulation—thereby providing an effective and efficient method of building viscosity without requiring large amounts of gum to achieve desired viscosities.

While the invention has been described with respect to specific examples including presently preferred modes of carrying out the invention, those skilled in the art will appreciate that there are numerous variations and permutations of the above described systems and techniques. It is to be understood that other embodiments may be utilized and structural and functional modifications may be made without departing from the scope of the present invention. Thus, the spirit and scope of the invention should be construed broadly as set forth in the appended claims.

What is claimed is:

1. A viscosity control composition comprising:
a surfactant blend comprising:
a biosurfactant;
an anionic surfactant;
a zwitterionic surfactant comprising sodium lauroamphoacetate;
a gum comprising carrageenan; and
a liquid carrier.

2. The viscosity control composition according to claim 1, wherein the biosurfactant comprises rhamnolipid.

3. The viscosity control composition according to claim 1, wherein the biosurfactant is present in an amount ranging from about 0.01 wt. % to about 13 wt. % based on the weight of the viscosity control composition.

4. The viscosity control composition according to claim 1, wherein the anionic surfactant comprises an amine-containing compound.

5. The viscosity control composition according to claim 1, wherein the anionic surfactant comprises sodium lauroyl sarcosinate.

6. The viscosity control composition according to claim 1, wherein the anionic surfactant is present in an amount ranging from about 0.1 wt. % to about 13.0 wt. % based on the weight of the viscosity control composition.

7. The viscosity control composition according to claim 1, wherein the zwitterionic surfactant is present in an amount ranging from about 1.0 wt. % to about 24.0 wt. % based on the weight of the viscosity control composition.

8. The viscosity control composition according to claim 1, wherein the gum is present in an amount ranging from about 0.1 wt. % to about 5.0 wt. % based on the weight of the viscosity control composition.

9. The viscosity control composition according to claim 1, wherein the gum consists of carrageenan.

10. The viscosity control composition according to claim 1, wherein the anionic surfactant and the biosurfactant are present in a weight ratio ranging from about 0.6:1 to about 2:1.

11. The viscosity control composition according to claim 1, wherein the zwitterionic surfactant and the biosurfactant are present in a weight ratio ranging from about 5:1 to about 10:1.

12. The viscosity control composition according to claim 1, wherein the gum and the biosurfactant are present in a weight ratio ranging from about 0.5:1 to about 2:1.

13. The viscosity control composition according to claim 1, wherein the liquid carrier comprises water.

14. The viscosity control composition according to claim 1, wherein the surfactant blend, the gum, and the liquid carrier total to about 100 wt. % of the viscosity control composition.

15. A viscosity control composition comprising:
a surfactant blend comprising:
a biosurfactant;
an anionic surfactant;
a zwitterionic surfactant;
a gum comprising carrageenan; and
a liquid carrier;
wherein the anionic surfactant and the biosurfactant are present in a weight ratio ranging from about 0.6:1 to about 2:1; wherein the zwitterionic surfactant and the biosurfactant are present in a weight ratio ranging from about 5:1 to about 10:1; and wherein the gum and the biosurfactant are present in a weight ratio ranging from about 0.5:1 to about 2:1.

16. A viscosity control composition comprising:
a rhamnolipid;
sodium lauroyl sarcosinate;
sodium lauroamphoacetate;
carrageenan; and
a liquid carrier.

17. The viscosity control composition according to claim 16, wherein the rhamnolipid, the sodium lauroyl sarcosinate, the sodium lauroamphoacetate, the carrageenan, and the liquid carrier total to about 100 wt. % of the viscosity control composition.

18. A personal care composition comprising:
the viscosity control composition according to claim 1; and
a personal care ingredient;
wherein the personal care composition is a bath soap, shower gel, shampoo, conditioner, body wash, or skin cleanser.

19. The personal care composition according to claim 18, wherein the personal care ingredient comprises one or more of a dye, fragrance, and combinations thereof.

20. A method of manufacturing a personal care product having viscosity control, the method comprising:
blending together a viscosity control composition according to claim 1, with a personal care ingredient to form a personal care composition; and
adding a predetermined volume of the personal care composition to a container to form the personal care product.
